(19) United States
(12) Patent Application Publication
(10) Pub. No.: US 2025/0320503 A1
(43) Pub. Date: Oct. 16, 2025

(54) AAV VECTORS ENCODING SOD1-TARGETING ARTIFICIAL MIRNAS (AMI-RNA)

(71) Applicant: University of Massachusetts, Westborough, MA (US)

(72) Inventors: Jun Xie, Worcester, MA (US); Guangping Gao, Worcester, MA (US); Zuoshang Xu, Worcester, MA (US); Hong Ma, Worcester, MA (US); Fang Wan, Worcester, MA (US)

(73) Assignee: University of Massachusetts, Westborough, MA (US)

(21) Appl. No.: 19/248,719

(22) Filed: Jun. 25, 2025

Related U.S. Application Data
(63) Continuation of application No. PCT/US2024/051348, filed on Oct. 15, 2024.
(60) Provisional application No. 63/590,724, filed on Oct. 16, 2023.

(51) Int. Cl. C12N 15/113; A61K 9/00; A61P 25/28; C07K 14/005; C12N 15/86

(52) U.S. Cl. CPC C12N 15/1137; A61K 9/0019; A61P 25/28; C07K 14/005; C12N 15/86; C12N 2310/141; C12N 2310/3519; C12N 2320/32; C12N 2750/14122; C12N 2750/14143; C12N 2830/50; C12Y 115/01001

(57) ABSTRACT

Aspects of the disclosure relate to compositions and methods for reducing expression or activity of superoxide dismutase 1 (SOD1) in a cell or subject. In some embodiments, the compositions, such as nucleic acid and viral vectors, comprise artificial microRNAs (amiRNAs) having a SOD1-targeting sequence positioned within a microRNA scaffold. In some embodiments, the compositions further comprise a human SMN1 promoter. In some aspects, the methods comprise administering a composition of the disclosure to a subject, for example a subject having amyotrophic lateral sclerosis (ALS).

### RELATED APPLICATIONS

This application claims priority under 35 U.S.C. § 119(e) to U.S. Provisional Patent Application Ser. No. 63/590,724, filed Oct. 16, 2023, and entitled “AAV VECTORS ENCODING SOD1-TARGETING ARTIFICIAL MIRNAS (AMI-RNA),” which is herein incorporated by reference in its entirety.

### REFERENCE TO AN ELECTRONIC SEQUENCE LISTING

The contents of the electronic sequence listing (U012070195WO00-SEQ-KZM.xml; Size: 19,618 bytes; and Date of Creation: Oct. 15, 2024) is herein incorporated by reference in its entirety.

### BACKGROUND

Autosomal dominant mutations in superoxide dismutase 1 (SOD1) gene cause motor neuron degeneration and are linked to 10-20% of familial- and ˜2% of sporadic Amyotrophic lateral sclerosis (ALS), a fatal disease for which an effective treatment is urgently need. Gene silencing of SOD1 using antisense oligonucleotides (ASO) or short interfering RNA (siRNA) has shown modest clinical benefits but requires repeated dosing to achieve therapeutic levels of knockdown.

### SUMMARY

Aspects of the disclosure relate to compositions and methods for silencing a SOD1 gene. The disclosure is based, in part, on nucleic acids (e.g., rAAV vectors) encoding artificial microRNAs (amiRNAs) comprising a miR-33 pri-miRNA scaffold and a nucleotide sequence (e.g., a guide strand) targeting SOD1. In some embodiments, expression of the inhibitory nucleic acid is driven by a neuron-specific promoter, for example a survival motor neuron (SMN1) promoter. In some embodiments, compositions described by the disclosure are useful for inhibiting SOD1 expression in a cell or subject having amyotrophic lateral sclerosis (ALS).

Accordingly, in some aspects, the disclosure provides a recombinant adeno-associated virus (rAAV) vector comprising a transgene comprising a human SMN1 promoter operably linked to a nucleic acid sequence encoding an artificial microRNA (amiRNA) targeting human SOD1, flanked by adeno-associated virus (AAV) inverted terminal repeats (ITRs).

In some embodiments, a human SMN1 promoter comprises a nucleic acid sequence that is at least 70%, 80%, 90%, 95%, or 99% identical to the nucleic acid sequence set forth in any one of SEQ ID NOs: 3-7. In some embodiments, an endogenous SMN1 promoter comprises or consists of the nucleic acid sequence set forth in SEQ ID NO: 4.

In some embodiments, an amiRNA comprises a miR-33 prim-miRNA scaffold; and a guide strand targeting a human SOD1 RNA transcript. In some embodiments, an amiRNA comprises or consists of the sequence set forth in SEQ ID NO: 1 or SEQ ID NO: 2.

In some embodiments, a transgene further comprises one or more miRNA binding sites.

In some embodiments, AAV ITRs are AAV2 ITRs. In some embodiments, at least one of the AAV ITRs is a mutant ITR (mTR).

In some aspects, the disclosure provides a recombinant adeno-associated virus (rAAV) comprising an rAAV vector comprising a transgene comprising a human SMN1 promoter operably linked to a nucleic acid sequence encoding an artificial microRNA (amiRNA) targeting human SOD1, flanked by adeno-associated virus (AAV) inverted terminal repeats (ITRs); and at least one AAV capsid protein.

In some embodiments, at least one AAV capsid protein is selected from an AAV2, AAV3, AAV4, AAV5, AAV6, AAV7, AAV8, or AAV9 capsid protein, or a variant thereof. In some embodiments, at least one AAV capsid protein is an AAV9 capsid protein. In some embodiments, the rAAV is a self-complementary AAV (scAAV).

In some embodiments, the disclosure provides a pharmaceutical composition comprising an rAAV vector or rAAV as described herein, and a pharmaceutically acceptable excipient.

In some aspects, the disclosure provides a method for delivering a transgene to a cell, the method comprising administering an rAAV vector or rAAV as described herein to a cell.

In some aspects, the disclosure provides a method for preventing or treating amyotrophic lateral sclerosis (ALS) in a subject, the method comprising administering an rAAV vector or rAAV as described herein to the subject.

In some embodiments, a cell is a mammalian cell. In some embodiments, a cell is a human cell. In some embodiments, a cell is a central nervous system (CNS) cell, optionally a neuronal cell.

In some embodiments, a cell is in a subject. In some embodiments, a subject has or is suspected of having amyotrophic lateral sclerosis (ALS). In some embodiments, a subject comprises a G93A mutation in a SOD1 gene.

In some embodiments, administering comprises systemic injection or local injection. In some embodiment, systemic injection comprises intravenous injection. In some embodiments, administering comprises injection to the central nervous system (CNS) of a subject.

In some aspects, the disclosure provides an isolated nucleic acid comprising the nucleic acid sequence set forth in any one of SEQ ID NOs: 1 to 10. In some aspects, the disclosure provides a vector comprising an isolated nucleic acid as described herein. In some embodiments, a vector is a plasmid or a baculovirus vector. In some aspects, the disclosure provides a host cell comprising an isolated nucleic acid or vector as described herein.

### BRIEF DESCRIPTION OF DRAWINGS

FIG. 1 is a schematic depicting SOD-1 targeting rAAV vectors as described by the disclosure.

FIG. 2 shows representative survival data after treatment with SOD1-targeting rAAV vectors in a mouse model of ALS. Median survival of PBS-injected SOD1-G93A mice was 117 days (n=17). Median survival of mice treated with CMVen/CB-amiR vector (n=5) was extended by 23 days. Median survival of the hSMN1-amiR (n=16) and KSMN1-dual-amiR (n=17) treatment groups were extended by 42 days and 98 days, respectively.

FIG. 3 shows representative qPCR data for SOD1 knockdown in selected tissues. Knockdown efficiencies in the hSMN1-dual-amiR group were 45% in the brainstem, 35-45% knockdown throughout the cervical and lumbar spinal cord, 83% in the quadriceps, 90% in the heart, and 98% in the liver.

FIGS. 4A-4F illustrate that silencing SOD1 expression improved rotarod latency and muscle strength, in accordance with some embodiments, hSOD1^{G93A} mice were injected with AAV9-hSMN1-dual-amiR or PBS at day 60, day 90, or day 105-120 at 1.0×10¹⁴ vg/kg by tail vein. Age-matched wild-type littermates were used as controls. Rotarod latency (FIGS. 4A-4C) and four limbs force (FIGS. 4D-4F) were measured at indicated time points in different animal cohorts.

FIGS. 5A-5B illustrate results of animal breath quantification using whole-body plethysmography on day 105 (FIG. 5A) and day 200 (FIG. 5B), in accordance with some embodiments, hSOD1^{G93A} mice were treated with AAV9-dual-amiR vector or PBS at day 60 and tested on day 105 and day 200. RA 01, Baseline; Gas 01, Hypercapnia (Increased CO₂); RA 02, Recovery Phases.

### DETAILED DESCRIPTION

Aspects of the disclosure relate to compositions and methods for silencing RNA transcripts expressed from a SOD1 gene. The disclosure is based, in part, on nucleic acids (e.g., rAAV vectors) encoding artificial microRNAs (amiRNAs) comprising a miR-33 pri-miRNA scaffold and a nucleotide sequence (e.g., a guide strand) targeting SOD1. In some embodiments, expression of the inhibitory nucleic acid is driven by a neuron-specific promoter, for example a survival motor neuron (SMN1) promoter. As described further in the Examples, administration of rAAV vectors comprising a combination of SMN1 promoter and amiRNAs targeting SOD1 surprisingly resulted in knockdown of endogenous SOD1 expression and extended survival of subjects having ALS (e.g., subjects having ALS characterized by a G93A mutation in a I gene). In some embodiments, compositions described by the disclosure are useful for inhibiting SOD1 expression in a cell or subject having amyotrophic lateral sclerosis (ALS).

### Nucleic Acids

Aspects of the disclosure relate to isolated nucleic acids encoding a transgene engineered to express one or more (e.g., 1, 2, 3, 4, 5, or more) inhibitory nucleic acids (e.g., an inhibitory RNA, such as an artificial miRNA, amiRNA). The one or more inhibitory nucleic acids may target (e.g., hybridize or specifically bind to) the same gene (e.g., hybridize or specifically bind to different sequences of the same gene) or different genes (e.g., hybridize or specifically bind to different genes).

A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

In some embodiments, any one or more thymidine (T) nucleotides or uridine (U) nucleotides in a sequence provided herein may be replaced with any other nucleotide suitable for base pairing (e.g., via a Watson-Crick base pair) with an adenosine nucleotide. For example, T may be replaced with U, and U may be replaced with T.

Inhibitory nucleic acids are small, non-coding RNAs that mediate gene silencing by various mechanisms. In some embodiments, an inhibitory RNA forms a hairpin structure. Generally, hairpin-forming RNAs are arranged into a self-complementary “stem-loop” structure that includes a single nucleic acid encoding a stem portion having a duplex comprising a sense strand (e.g., passenger strand) connected to an antisense strand (e.g., guide strand) by a loop sequence. The passenger strand and the guide strand share complementarity, In some embodiments, the passenger strand and guide strand share 100% complementarity. In some embodiments, the passenger strand and guide strand share at least 50%, at least 60%, at least 709%, at least 80%, at least 90%, at least 95%, or at least 99% complementarity. A passenger strand and a guide strand may lack complementarity due to a base-pair mismatch. In some embodiments, the passenger strand and guide strand of a hairpin-forming RNA have at least 1, at least 2, at least 3, at least 4, at least 5, at least 6, at least 7 at least 8, at least 9, or at least 10 mismatches. Generally, the first 2-8 nucleotides of the stem (relative to the loop) are referred to as “seed” residues and play an important role in target recognition and binding. The first residue of the stem (relative to the loop) is referred to as the “anchor” residue. In some embodiments, hairpin-forming RNA have a mismatch at the anchor residue.

In some embodiments, an inhibitory RNA is processed in a cell (or subject) to form a “mature miRNA”. Mature miRNA is the result of a multistep pathway which is initiated through the transcription of primary miRNA from its miRNA gene or intron, by RNA polymerase II or III generating the initial precursor molecule in the biological pathway resulting in miRNA. Once transcribed, pri-miRNA (often over a thousand nucleotides long with a hairpin structure) is processed by the Drosha enzyme which cleaves pri-miRNA near the junction between the hairpin structure and the ssRNA, resulting in precursor miRNA (pre-miRNA). The pre-miRNA is exported to the cytoplasm where is further reduced by Dicer enzyme at the pre-miRNA loop, resulting in duplexed miRNA strands.

Of the two strands of a miRNA duplex, one arm, the guide strand (miR), is typically found in higher concentrations and binds and associates with the Argonante protein which is eventually loaded into the RNA-inducing silencing complex (RISC). The guide strand miRNA-RISC complex helps regulates gene expression by binding to its complementary sequence of mRNA, often in the 3′ UTR of the mRNA. The non-guide strand of the miRNA duplex is known as the passenger strand and is often degraded but may persist and also act either intact or after partial degradation to have a functional role in gene expression.

In some embodiments, a transgene is engineered to express an inhibitory nucleic acid (e.g., an miRNA) having a guide strand that targets a human gene. “Targeting” refers to hybridization or specific binding of an inhibitory nucleic acid to its cognate (e.g., complementary) sequence on a target gene (e.g., mRNA transcript of a target gene). In some embodiments, an inhibitory nucleic acid that targets a gene shares a region of complementarity with the target gene that is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, a region of complementarity is more than 30 nucleotides in length.

In some embodiments, the guide strand targets a human gene associated with a disease or disorder, for example SOD1 (associated with amyotrophic lateral sclerosis, ALS). In some embodiments, a guide strand that targets SOD1 is encoded by an isolated nucleic acid comprising the sequence set forth in SEQ ID NO: 1 or SEQ ID NO: 2.

In some embodiments, the inhibitory nucleic acid is 5 to 300 bases in length (e.g., 10-30, 15-25, 19-22, 25-50, 40-90, 75-100, 90-150, 110-200, 150-250, 200-300, etc. nucleotides in length). The inhibitory nucleic acid sequence encoding a pre-miRNA or mature miRNA may be 10-50, or 5-50 bases length. In some embodiments, an inhibitory nucleic acid sequence comprising a pri-miRNA scaffold (and is at least 250, 260, 270, 280, 290, or 300 bases in length. In some embodiments, the inhibitory nucleic acid comprises or consists of a sequence of bases at least 80% or 90% complementary to, e.g., at least 5, 10, 15, 20, 25 or 30 bases of, or up to 30 or 40 bases of, a target nucleic acid (e.g., a human gene, such as SOD1), or comprises a sequence of bases with up to 3 mismatches (e.g., up to 1, or up to 2 mismatches) over 10, 15, 20, 25 or 30 bases of a target nucleic acid (e.g., a human gene, such as SOD1).

In some embodiments, an inhibitory nucleic acid is an artificial miRNA (amiRNA). An artificial microRNA (AmiRNA) is derived by modifying a native miRNA to replace natural targeting regions of pre-mRNA with a targeting region of interest. For example, a naturally occurring, expressed miRNA can be used as a scaffold or backbone (e.g., a pri-miRNA scaffold), with the stem sequence replaced by that of a miRNA targeting a gene of interest. An artificial precursor microRNA (pre-amiRNA) is normally processed such that one single stable small RNA is preferentially generated.

Aspects of the disclosure relate to a nucleic acid sequence encoding a guide strand targeting a human gene that is inserted in a pri-miRNA scaffold. In some embodiments, the pri-miRNA scaffold is a non-human (e.g., mouse) scaffold. In some embodiments, the pri-miRNA scaffold is a human scaffold. In some embodiments, a mouse pri-miRNA scaffold is selected from: pri-miR-122, pri-miR-33, pri-miR-26a, pri-miR-126, pri-miR-22, pri-miR-199, pri-miR-99, pri-miR-21, pri-miR-375, pri-miR-101, pri-miR-451, pri-miR-194, pri-miR-30a, and pri-miR-155. In some embodiments, a human pri-miRNA scaffold is selected from: pri-miR-122, pr-miR-33, pri-miR-26a, pri-miR-126, pri-miR-22, pri-miR-199, pri-miR-99, pri-miR-21, pri-miR-375, pri-miR-101, pri-miR-451, pri-miR-194, pri-miR-30a, and pri-miR-155. In some embodiments, the pri-miRNA is a mouse pri-miRNA-33 scaffold. In some embodiments, the pri-miRNA is a human pri-miRNA-33 scaffold. In some embodiments, the pri-miRNA scaffold flanks an inhibitory nucleic acid encoding SOD1. In some embodiments an amiRNA comprising a miR-33 scaffold and an inhibitory nucleic acid targeting SOD1 comprises the sequence set forth in SEQ ID NO: 1 or SEQ ID NO: 2.

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein and/or an expression control sequence (e.g., a poly-A tail), as described elsewhere in the disclosure. In some embodiments, the vector comprises: 1) An amiR embedded in a mouse miR-33 scaffold driven by the cytomegalovirus enhancer/chicken β-actin promoter (e.g., CMVen/CB-amiR); 2) an amiR embedded in a human miR-33 scaffold driven by a promoter derived from the endogenous human survival motor neuron 1 promoter (e.g., hSMN1-amiR, comprising a promoter as set forth in any one of SEQ ID NOs: 3-7); or 3) two amiRs embedded in mouse and human miR-33 scaffolds driven by the hSMN1 promoter (hSMN1-dual-amiR).

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol, 70:520 532 (1996). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are AAV2 ITRs. In some embodiments, at least one AAV ITR is a truncated AAV ITR (e.g., a mutant ITR, also referred to as an mTR), for example a ΔITR as described, for example by McCarty (2008) Molecular Therapy 16(10): 1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein of polypeptide. Similarly, two or more coding regions are operably linked when they are linked in such a way that their transcription from a common promoter results in the expression of two or more proteins having been translated in frame. In some embodiments, operably linked coding sequences yield a fusion protein.

A region comprising a transgene (e.g., a transgene encoding a SMN1 protein, etc.) may be positioned at any suitable location of the isolated nucleic acid that will enable expression of the at least one transgene, the selectable marker protein, or reporter protein.

It should be appreciated that in cases where a transgene encodes more than one gene product (e.g., a SMN1 protein and another protein or interfering nucleic acid), each gene product may be positioned in any suitable location within the transgene. For example, a nucleic acid encoding a first polypeptide may be positioned in an intron of the transgene and a nucleic acid sequence encoding a second polypeptide may be positioned in another untranslated region (e.g., between the last codon of a protein coding sequence and the first base of the poly-A signal of the transgene).

A “promoter” refers to a DNA sequence recognized by the synthetic machinery of the cell, or introduced synthetic machinery, required to initiate the specific transcription of a gene. The phrases “operatively linked,” “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the disclosure may also contain an intron, desirably located between the promoter/enhancer sequence and the transgene. One possible intron sequence is derived from SV-40, and is referred to as the SV-40 T intron sequence. In some embodiments, an intron is a non-native intron or synthetic intron (e.g., a MBL intron). Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional, and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furter, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Fadler, S et al., Gene Therapy, 2001; 8: 864-873; and Halpin, C et al., The Plant Journal, 1999; 4: 453-459; de Felipe, P et al., Gene Therapy, 1999; 6:198-208; de Felipe, P et al., Human Gene Therapy, 2000; 11: 1921-1931.; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is a chicken β-actin (CBA) promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor vins (MMTV) promoter, the T7 polymerase promoter system (WO 98/10088); the ecdysone insect promoter (No et al., Proc. Natl. Acad Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997). Still other types of inducible promoters which may be useful in this context are those which are regulated by a specific physiological state, e.g., temperature, acute phase, a particular differentiation state of the cell, or in replicating cells only.

Aspects of the disclosure relate to isolated nucleic acids and rAAV vectors comprising a nucleic acid sequence encoding an amiRNA targeting SOD1 operably linked to a native promoter. In some embodiments a native promoter comprises a human SMN1 promoter, or a variant thereof. In some embodiments, a human SMN1 promoter comprises the nucleic acid sequence set forth in any one of SEQ ID NOs: 3-7. In some embodiments, a human SMN1 promoter comprises the nucleic acid sequence set forth in SEQ ID NOs: 3. In some embodiments, a human SMN1 promoter comprises the nucleic acid sequence set forth in SEQ ID NOs: 4. In some embodiments, a human SMN1 promoter comprises the nucleic acid sequence set forth in SEQ ID NOs: 5. In some embodiments, a human SMN1 promoter comprises the nucleic acid sequence set forth in SEQ ID NOs: 6. In some embodiments, a human SMN1 promoter comprises the nucleic acid sequence set forth in SEQ ID NOs: 7.

In some embodiments, a human SMN1 promoter or variant thereof comprises a nucleic acid sequence that is at least 50%, at least 60%, at least 70%, at least 80%, at least 90%, at least 91%, at least 92%, at least 93%, at least 94%, at least 95%, at least 96%, at least 97%, at least 98%, at least 99%, or 100% identical to the nucleic acid sequence set forth in any one of SEQ ID NOs: 3-7. Human SMN1 promoters are generally known in the art, for example as described by Echaniz-Laguna et al., Am. J. Hum. Genet. 64; 1365-1370, 1999. The native promoter may be preferred when it is desired that expression of the transgene should mimic the native expression (e.g., express physiological levels of transgene, such as amiRNA targeting SOD1, in the appropriate cell types). The native promoter may be used when expression of the transgene must be regulated temporally or developmentally, or in a tissue-specific manner, or in response to specific transcriptional stimuli. Without wishing to be bound by any theory, use of a human SMN1 promoter in isolated nucleic acids and rAAV vectors described herein better regulates expression of amiRNAs from the vectors in neurons relative to expression of the amiRNAs from isolated nucleic acids and rAAV vectors comprising other promoters, for example CMV promoter, chicken-beta actin (CBA) promoter, CB6 promoter, etc. In a further embodiment, other native expression control elements, such as enhancer elements, polyadenylation sites, and/or Kozak consensus sequences may also be used to mimic native expression.

In some aspects, the disclosure relates to isolated nucleic acids comprising a transgene encoding one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in a 3′ untranslated region (3′ UTR) of a transgene, for example between the last codon of a nucleic acid sequence encoding one or more complement control proteins as described herein, and a poly A sequence.

In some embodiments, the rAAV vector comprises AAV inverted terminal repeats (ITRs). In some embodiments, the ITRs are AAV2 ITRs. In some embodiments, at least one of the ITRs is a mutant ITR (e.g., mTR), for example a delta ITR. In some embodiments, the rAAV vector is a self-complementary AAV (scAAV) vector. In some embodiments, the rAAV vector described herein comprises a nucleic acid sequence at least 60%, at least 70%, at least 80%, at least 90%, at least 91%, at least 92%, at least 93%, at least 94%, at least 95%, at least 96%, at least 97%, at least 98%, at least 99%, or 100% identical to any one of SEQ ID NOs: 8-10.

In some embodiments, transgene expression causes overexpression of the transgene in the liver, resulting in liver toxicity (see, e.g., Hinderer et al., Severe Toxicity in Nonhuman Primates and Piglets Following High-Dose Intravenous Administration of an Adeno-Associated Virus Vector Expressing Human SMN, Volume: 29 Issue 3, 285-298: Mar. 1, 2018. In some embodiments, in order to reduce liver toxicity, the AAV vector comprises a transgene comprises one or more (e.g., 1, 2, 3, 4, 5, or more) miRNA binding sites that de-target expression of a transgene from liver cells. For example, in some embodiments, a transgene comprises one or more miR-122 binding sites. In some embodiments, the rAAV vectors described herein comprise one or more miR-122 binding sites.

In some embodiments, a transgene comprises one or more (e.g., 1, 2, 3, 4, 5, or more) miRNA binding sites that de-target expression of a transgene from immune cells (e.g., antigen presenting cells (APCs), such as macrophages, dendrites, etc.). Incorporation of miRNA binding sites for immune-associated miRNAs may de-target transgene (e.g., one or more inhibitory nucleic acids) expression from antigen presenting cells and thus reduce or eliminate immune responses (cellular and/or humoral) produced in the subject against products of the transgene, for example as described in US 2018/0066279, the entire contents of which are incorporated herein by reference.

As used herein an “immune-associated miRNA” is an miRNA preferentially expressed in a cell of the immune system, such as an antigen presenting cell (APC). In some embodiments, an immune-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune-associated miRNA is expressed is a B cell, T cell, Killer T cell, Helper T cell, γδ T cell, dendritic cell, macrophage, monocyte, vascular endothelial cell, or other immune cell. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, OX40, PD-1 (Programmed death-1), RT6, TCR (T cell receptor), Thy-1 (CD90), and TSA-2 (Thymic shared Ag-2). In some embodiments, the immune-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152. In some embodiments, a transgene described herein comprises one or more binding sites for miR-122.

### Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Sach AAVs are referred to herein as “recombinant AAVs”. Recombinant AAVs (rAAVs) preferably have tissue-specific targeting capabilities, such that a transgene of the rAAV will be delivered specifically to one or more predetermined tissue(s) (e.g., muscle tissues, ocular tissues, neurons, etc.). The AAV capsid is an important element in determining these tissue-specific targeting capabilities (e.g., tissue tropism). Thus, an rAAV having a capsid appropriate for the tissue being targeted can be selected. In some embodiments, the rAAV comprises an AAV9 serotype capsid protein.

In some embodiments, rAAVs of the disclosure comprise the nucleotide sequence as set forth in any one of SEQ ID NO: 8-10, or encode one or more (e.g., 1, 2, 3, 4, 5, or more) amiRNA having the sequence as set forth in SEQ ID NO: 1 or SEQ ID NO: 2. In some embodiments, rAAVs of the disclosure comprise a nucleotide sequence that is 99% identical, 95% identical, 90% identical, 85% identical, 80% identical, 75% identical, 70% identical, 65% identical, 60% identical, 55% identical, or 50% identical to the nucleotide sequence as set forth in any one of SEQ ID NOs: 8-10.

In some aspects, the present disclosure provides a recombinant adeno-associated virus (rAAV) comprising: (a) a self-complementary rAAV genome comprising: (i) a 5′ ITR; (ii) a human SMN promoter comprising the nucleotide sequence of any one of SEQ ID NOs: 3-7; (iii) a nucleic acid sequence encoding one or more (e.g., 1, 2, 3, 4, 5, or more) SOD1-targeting amiRNA comprising the nucleic acid sequence set forth in SEQ ID NO: 1 or SEQ ID NO: 2; and (iv) a 3′ ITR; and (b) a AAV9 capsid protein. In some embodiments, the rAAV further comprises a poly A tail, such as a rabbit globin poly A or a BGH poly A tail. In some embodiments, the rAAV further comprises one or more miR-122 binding sites.

Methods for obtaining recombinant AAVs having a desired capsid protein are well known in the art. (See, for example, US 2003/0138772), the contents of which are incorporated herein by reference in their entirety). Typically, the methods involve culturing a host cell which contains a nucleic acid sequence encoding an AAV capsid protein; a functional rep gene; a recombinant AAV vector composed of AAV inverted terminal repeats (TTRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein has a tropism for central nervous system (CNS) tissues. In some embodiments, an AAV capsid protein targets neuronal cell types, astrocytes, oligodendrocytes, glial cells, etc. In some embodiments, an AAV capsid protein is of an AAV serotype selected from the group consisting of AAV1, AAV2, AAV3, AAV4, AAV5, AAV6, AAV7, AAV8, AAVrh8, AAV9, AAV10, AAVch10, AAV.PHP-eB, AAVrh39, AAVrh43, and variants of any of the foregoing. In some embodiments, the rAAV comprises an AAV9 capsid protein.

In some embodiments, an rAAV vector or rAAV particle comprises a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector (scAAV or scrAAV vector), for example as described by McCarthy (2008) Molecular Therapy 16(10):1648-1656.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a codon-optimized coding sequence encoding a transgene (e.g., SMN1). A “host cell” refers to any cell that harbors, or is capable of harboring, a substance of interest. Often a host cell is a mammalian cell. In some embodiments, a host cell is a neuron. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a central nervous system cell, for example a neuron or a glial cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual. Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, the term includes cloning and expression vehicles, as well as viral vectors. In some embodiments a vector comprises a baculovirus vector, which are useful for producing viral particles in certain insect cells (e.g., SF9 cells). In some embodiments, useful vectors are contemplated to be those vectors in which the nucleic acid segment to be transcribed is positioned under the transcriptional control of a promoter.

### Delivery of a Transgene to Tissue

In some aspects, inhibitory nucleic acids described herein are useful for inhibiting (e.g., reducing or silencing) expression of a target gene (e.g., mRNA transcript of a target gene) in a cell or subject. A “target gene” generally refers to a gene the expression of which it is desirable to inhibit. In some embodiments, a target gene expresses a mutant protein or protein associated with a disease or disorder. In some embodiments, the target gene is a gene associated with a neurodegenerative disease (e.g., ALS, Huntington's disease, Canavan disease, Alzheimer's disease, etc.). In some embodiments, the target gene is SOD1.

In some embodiments, administration of isolated nucleic acids and rAAVs described herein to a cell or subject results in inhibition of target gene expression in the cell or subject (e.g., inhibition relative to the level of target gene expression prior to the administration, or relative to a healthy control subject). In some embodiments, administration results in inhibition of target gene expression of at least 2-fold, 3-fold, 4-fold, 5-fold, 10-fold, 50-fold, 100-fold, or 1000-fold. In some embodiments, administration results in inhibition of target gene expression more than 1000-fold. In some embodiments, administration results in inhibition of target gene expression of at least 50%, 60%, 70%, 80%, 90%, 95%, or 99%. In some embodiments, administration results in complete (e.g., 100%, or no expression) inhibition of target gene expression.

The isolated nucleic acids, rAAVs, and compositions of the disclosure may be delivered to a subject in compositions according to any appropriate methods known in the art. For example, an rAAV, preferably suspended in a physiologically compatible carrier (e.g., in a composition), may be administered to a subject, i.e, host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human. In some embodiments, a subject is human.

Delivery of the rAAVs may be by, for example intramuscular injection or infusion into the muscle tissue or cells of a subject. As used herein, “muscle tissues” refers to any tissue derived from or contained in skeletal muscle, smooth muscle, or cardiac muscle of a subject. Non-limiting examples of muscle tissues include skeletal muscle, smooth muscle, cardiac muscle, myocytes, sarcomeres, myofibrils, etc.

Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue.

Aspects of the instant disclosure relate to compositions comprising a recombinant AAV comprising a capsid protein and a nucleic acid encoding a transgene, wherein the transgene comprises an amiRNA targeting a SOD1 RNA transcript. In some embodiments, the nucleic acid further comprises AAV ITRs. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, the compositions of the disclosure may contain, in addition to the rAAV and carrier(s), other conventional pharmaceutical ingredients, such as preservatives, of chemical stabilizers. Suitable exemplary preservatives include chlorobutanol, potassium sorbate, sorbic acid, sulfur dioxide, propyl gallate, the parabens, ethyl vanillin, glycerin, phenol. parachlorophenol, and poloxamers (non-ionic surfactants) such as Pluronic® F-68. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), intraocular injection, subretinal injection, oral, inhalation (including intranasal and intratracheal delivery), intravenous, intramuscular, subcutaneous, intradermal, iotratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art. In some embodiments, an rAAV as described herein is administered to a subject in a dose ranging between about 1 ml to about 100 ml of solution containing from about 10⁹ to 10¹⁶ genome copies. In some cases, a dosage between about 10¹¹ to 10¹³ rAAV genome copies is administered.

An effective amount of an rAAV is an amount sufficient to target infect an animal, target a desired tissue. In some embodiments, an effective amount of an rAAV is administered to the subject during a pre-symptomatic stage of degenerative disease. In some embodiments, a subject is administered an rAAV or composition after exhibiting one or more signs or symptoms of degenerative disease.

An effective amount of an rAAV may also depend on the mode of administration. For example, targeting a muscle tissue (e.g., muscle cells) by intramuscular administration or subcutaneous injection may require different (e.g., higher or lower) doses, in some cases, than targeting muscle tissue by another method (e.g., systemic administration, topical administration, etc.). In some embodiments, the injection is systemic administration (e.g., intravenous injection). In some cases, multiple doses of a rAAV are administered. In some embodiments, the administration is systemic administration. In some embodiments, the systemic administration comprises intravenous administration. In some embodiments, the administration is local administration to the central nervous system, for example by intracerebral injection, intrathecal injection, intracranial injection, etc.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either intraocularly, subretinally, subcutaneously, intraopancreatically, intranasally, parenterally, intravenously, intramuscularly, intrathecally, orally, intraperitoneally, or by inhalation. In some embodiments, the administration modalities as described in U S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringeability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropnate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the disclosure into suitable host cells. In particular, the rAAV vector delivered transgenes may be formulated for delivery either encapsulated in a lipid particle, a liposome, a vesicle, a nanosphere, or a nanoparticle or the like.

Sach formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles false termed maltilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708) microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Therapeutic Methods

In some aspects, inhibitory nucleic acids described herein are useful for inhibiting (e.g., reducing or silencing) expression of a target gene (e.g., mRNA transcript of a target gene) in a cell or subject. In some embodiments, a target gene expresses a mutant protein or protein associated with a disease or disorder. In some embodiments, the target gene is a gene associated with a neurodegenerative disease (e.g., ALS, Huntington's disease, Canavan disease, Alzheimer's disease, etc.). In some embodiments, the target gene is SOD1.

In some embodiments, administration of isolated nucleic acids and rAAVs described herein to a cell or subject results in inhibition of target gene expression in the cell or subject (e.g., inhibition relative to the level of target gene expression prior to the administration, or relative to a healthy control subject). In some embodiments, administration results in inhibition of target gene expression of at least 2-fold, 3-fold, 4-fold, 5-fold, 10-fold, 50-fold, 100-fold, or 1000-fold. In some embodiments, administration results in inhibition of target gene expression more than 1000-fold. In some embodiments, administration results in inhibition of target gene expression of at least 50%, 60%, 70%, 80%, 90%, 95%, or 99%. In some embodiments, administration results in complete (e.g., 100%, or no expression) inhibition of target gene expression.

Methods for delivering a transgene (e.g., an isolated nucleic acid or rAAV engineered to express one or more inhibitory nucleic acids as described herein) to a cell or a subject are provided by the disclosure. The methods typically involve administering to a subject an effective amount of an isolated nucleic acid encoding the transgene(s). In some embodiments, expression constructs described by the disclosure are useful for treating diseases, such as ALS.

In some aspects the disclosure relates to a method of treating amyotrophic lateral sclerosis (ALS) in a subject, the method comprising administering to a subject in need thereof an effective amount of an isolated nucleic acid or an rAAV as described herein. A subject may be any mammalian organism, for example a human, non-human primate, horse, pig, dog, cat rodent, etc. In some embodiments a subject is a human.

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, a target tissue is liver tissue (e.g., hepatocytes, neurons, etc.). In some embodiments, an effective amount of an isolated nucleic acid (e.g., which may be delivered via an rAAV) may be an amount sufficient to have a therapeutic benefit in a subject, e.g., to decrease the expression of one or more genes associated with ALS (e.g., SOD1), to extend the lifespan of a subject, to improve in the subject one or more symptoms of disease (e.g., a symptom of ALS or obesity), etc. The effective amount will depend on a variety of factors such as, for example, the species, age, weight, health of the subject, and the tissue to be targeted, and may thus vary among subject and tissue as described elsewhere in the disclosure.

In some embodiments, the term “treating” refers to the application or administration of a composition encoding a transgene(s) to a subject, who has ALS, a symptom of ALS, or a predisposition toward ALS (e.g., one or more mutations in a SOD1 gene, etc.), with the purpose to cure, heal, alleviate, relieve, alter, remedy, ameliorate, improve, or affect the disorder, the symptom of the disease, or the predisposition toward ALS.

Alleviating disease (e.g., ALS) includes delaying the development or progression of the disease, or reducing disease severity. Alleviating the disease does not necessarily require curative results. As used therein, “delaying” the development of a disease (such as ALS) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

In some embodiments, treating or delaying development of ALS comprises: extending survival, motor function (e.g., delaying limb paralysis), and muscle strength, delaying body weight loss; and/or delaying decline in respiratory capacity.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of ALS includes initial onset and/or recurrence.

In some embodiments, administration occurs via systemic injection or direct injection to the CNS. In some embodiments, systemic injection is intravenous injection.

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to an animal, such as a syringe, topical application devices, or intravenous needle tubing and bag, particularly in the case of the kits for producing specific somatic animal models.

The kit may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for constructing an AAV vector as described herein. In addition, kits of the disclosure may include, instructions, a negative and/or positive control, containers, diluents and buffers for the sample, sample preparation tubes and a printed or electronic table of reference AAV sequence for sequence comparisons.

### EXAMPLES

### Example 1

Amyotrophic lateral sclerosis (ALS) is a progressive neurodegenerative disease caused by motor neuron death. Autosomal dominant mutations in the superoxide dismutase 1 (SOD1) gene contribute to ˜2% of all ALS cases. Artificial microRNAs (amiRs) designed to target Sod1 and delivered by adeno-associated virus (AAV) vectors are promising approach for mitigating disease progression.

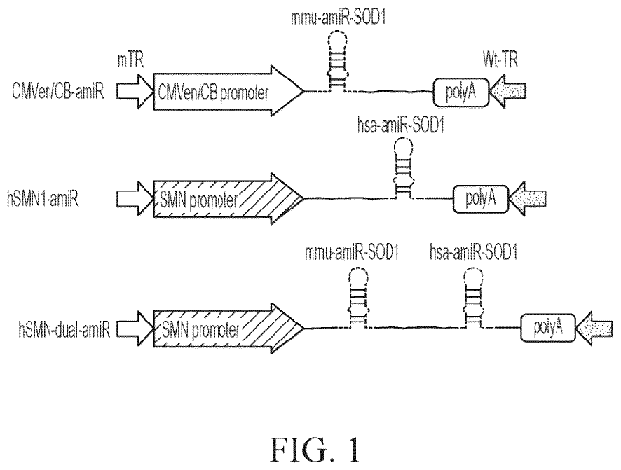

This example describes rAAV vectors encoding artificial microRNAs (amiRs) targeting SOD1. Briefly, rAAV vectors comprising a miR-33 amiRNA scaffold and a nucleic acid sequence targeting SOD1 were packaged into AAV9 particles. Three vector constructs were generated for this example: 1) An amiR embedded in a mouse miR-33 scaffold driven by the cytomegalovirus enhancer/chicken β-actin promoter (CMVen/CB-amiR); 2) an amiR embedded in a human miR-33 scaffold driven by a promoter derived from the endogenous human survival motor neuron 1 (hSMN1-amiR) promoter; and 3) two amiRs embedded in mouse and human miR-33 scaffolds driven by the hSMN1 promoter (hSMN1-dual-amiR), as shown in FIG. 1.

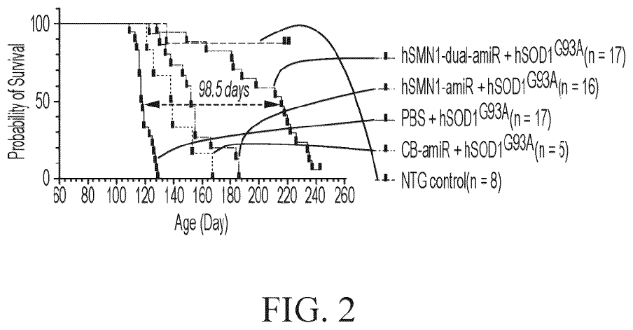

The three AAV9-amiR vectors were injected intravenously into SOD1-G93A mice at 60-68 days of age at a dose of 1.0×10¹⁴ vg/kg by tail vein in separate test groups and observed. PBS-treated transgenic animals and the age-matched non-transgenic litter mates were used as controls. The median survival of PBS-injected SOD1-G93A mice was 117 days (n=17). The median survival of mice treated with the CMVen/CB-amiR vector (n=5) was extended by 23days. Strikingly, the median survival of the hSMN1-amiR (n=16) and hSMN1-dual-amiR (n=17) treatment groups were extended by 42 days and 98 days, respectively (FIG. 2).

On day 105, ventilation was quantified using whole-body plethysmography to gauge diaphragm function and breathing. When compared to age-matched wildtype nice. PBS-treated SOD1-G93A mice showed response drops to hypercapnia in minute ventilation, and peak expiration and inspiratory flows. In contrast, hSMN1-dual-amiR vector-treated SOD1-G93A mice performed similarly with wildtype mice. On day 190, both rotarod and grip tests showed no difference between the hSMN1-dual-amiR group and age-matched wildtype mice.

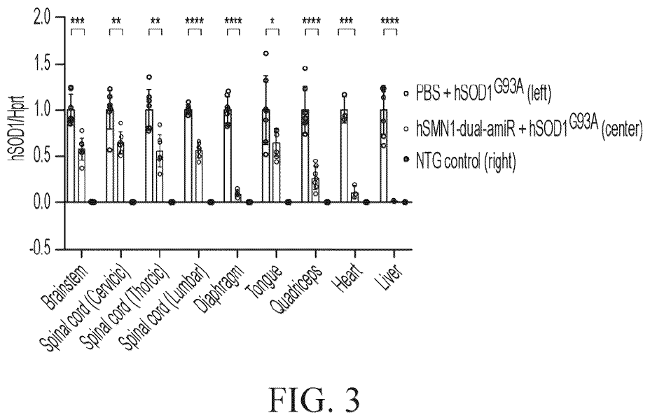

Measurements of SOD1 knockdown by qRT-PCR analysis were performed with tissues collected on day 105 (FIG. 3). Observed knockdown efficiencies in the hSMN1-dual-amiR group were 45% in the brainstem, 35-45% knockdown throughout the cervical and lumbar spinal cord, 83% in the quadriceps, 90% in the heart, and 98% in the liver. In summary, a single intravascular injection of AAV9-hSMN1-dual-amiR vector silences SOD1 expression within CNS and non-CNS tissues, efficiently preserves respiration and motor function, and drastically extends the survival by 98 days.

### Example 2

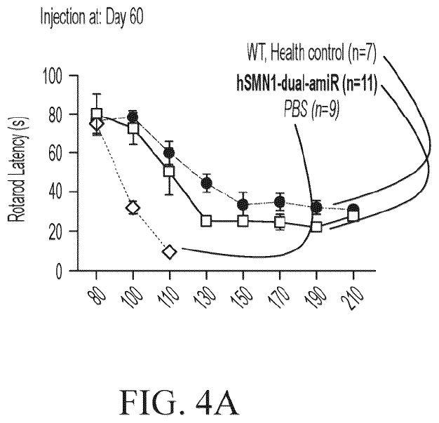

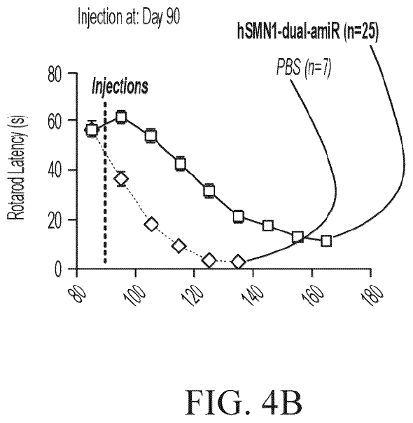

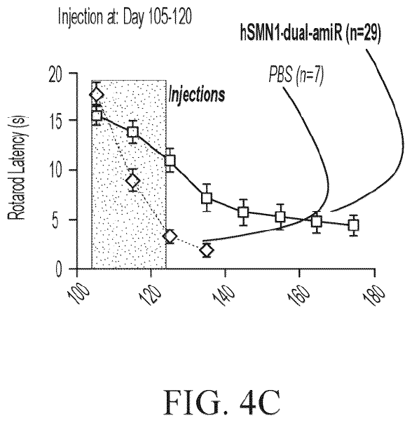

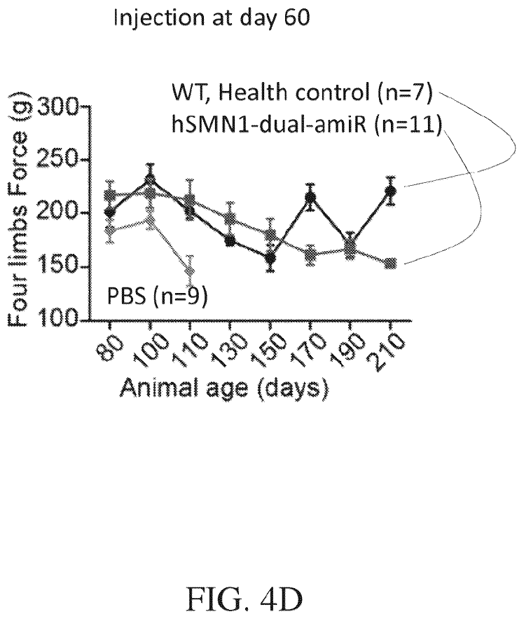

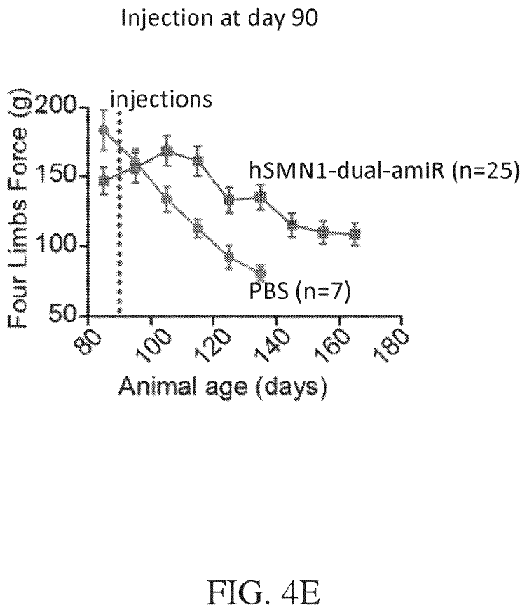

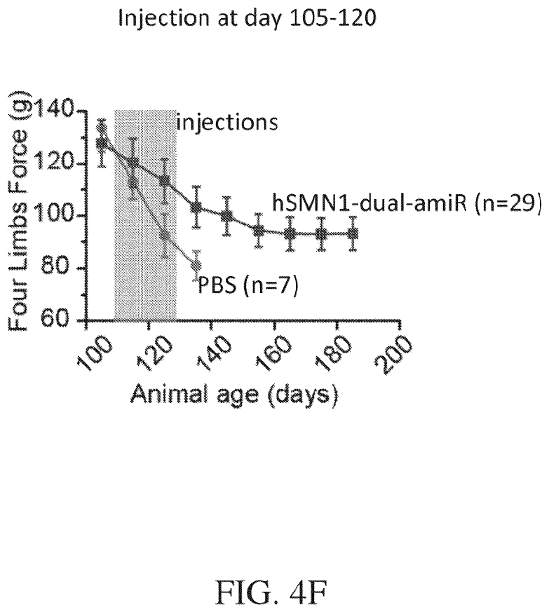

AAV9 hSMN1-dual-amiR were injected into SOD1^{G93A} mice by tail vein at the dose of 1.0×10¹⁴ vg/kg at three different time points: day 60-68 (the asymptomatic stage), day 90 (the non-motor symptomatic stage when mice start to lose body weight), and day 105-125 (the motor symptomatic stage). PBS-injected SOD1^{G93A} mice were used as a control to compare the therapeutic outcomes, including respiration function, muscle strength, and motor function. In addition to observing the mice with limb paralysis and body weight, motor function and muscle strength were also assessed in the AAV9-hSMN1-dual-amiR or PBS-treated SOD1^{G93A} mice every 10 or 20 days until the humane endpoint of animals. Wild-type littermates were used as controls. The motor function assessed by rotarod test was significantly preserved by the AAV9-hSMN1-dual-amiR treatment regardless of the time of treatment, compared to the PBS group in all three different time points (FIGS. 4A-4C). Strikingly, the diseased mice (n=11; 4 male and 7 female) performed similarly to their littermates (n=9; 5 male and 4 female) until day 210, when they were treated on day 60 (FIG. 4A). The treatment also preserved the four limbs' strength until day 190, when they were treated on day 60 (FIG. 4D). The AAV treatment after disease onset (n=25; 15 male and 10 female at day 90 in the vector treatment group, n=29; 16 male and 13 female at day 105-120 in the vector treatment group; n=7, 4 male and 3 female in the PBS group) still slowed down the declining of muscle strength (FIGS. 4E-4F). A slight increase of four-limb strength was even observed after treatment on the day 90 treated mice (FIG. 4E). Notably, about 20-30% of AAV9-hSMN1-dual-amiR treated mice were euthanized because of urination issues, not due to limb paralysis or body weight loss, which are the humane endpoints (data not shown). Taken together, this data showed early treatment correlated with more therapeutic benefits on survival, disease progression, motor function, and muscle strength, which is the consensus on the therapeutic window for ALS treatment. More importantly, the AAV9-hSMN1-dual-amiR vector preserved motor function and muscle strength after the onset of ALS (FIGS. 4B-4C and FIGS. 4B-4F).

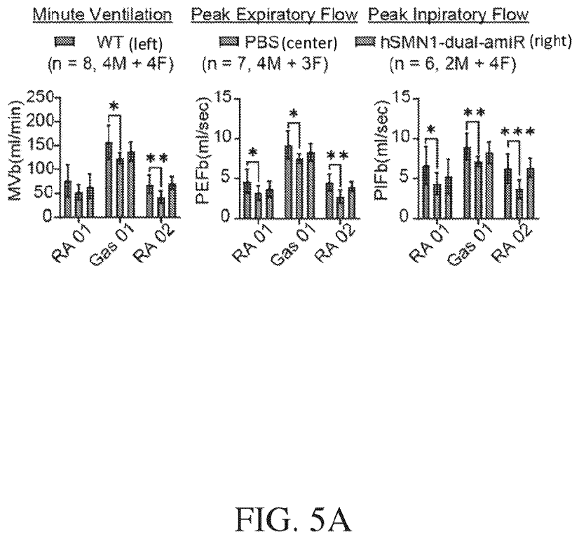

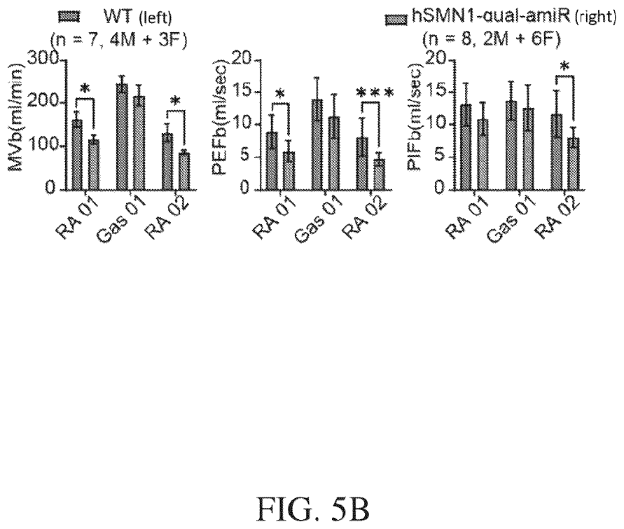

Respiratory failure is generally regarded as the most frequent cause of ALS patient death. The SOD1^{G93A} mouse model had significant respiratory insufficiency, restrictive lung disease, and hypoventilation. To gauge the animal breath function on day 105 and day 200, ventilation was quantified using whole-body plethysmography on mice treated on day 60. When compared to age-matched litter mates at day 105, PBS-treated SOD1^{G93A} mice showed compromised respiratory function in minute ventilation after the CO₂ challenge and during recovery. Peak expiration and inspiratory flows on the baseline, CO₂ challenging, and recovery phases were all impaired in disease animals compared to the healthy controls. In contrast, AAV9-hSMN1-dual-amiR vector-treated SOD1^{G93A} mice performed at the same level as the age-matched littermates (FIG. 5A). On day 200, the overall respiratory capacity declined in the treated SOD1^{G93A} mice. Still, the peak inspiratory flow remained comparable to their littermates at baseline. All these three parameters (MVb, PEFb, and PIFb) were still undistinguished from the littermates during the CO₂ challenge (FIG. 5B).

REPRESENTATIVE SEQUENCES
>Haman miR-33 scaffold with SOD1-targeting
sequence (SEQ ID NO: 1)
gatctGGCAGCCTTGGAGTGGGTTCCTGCCCCCTCGGGCACACAA
ACAGAGCTGAAGACCACCCTGGGCACCTCCTTGGCTGGCCGCATA
CCTCCTGGCGGGCAGCTGTGttctgctcgaaattgatgalgTGTT
CTGGTGGTACCCACATCATCATATCCGAGCAGAACACAGAGGCCT
GCCTGGCCCTCGAGAGACTGCCCTGACTGAAGGCCCTATCAGGTG
GGGGAGGGGATCCTGATAGAGGGCACTGCTGCCACTGTTGGGGCC
CAAGCtgca
>Mouse miR-33 scaffold with SOD1-targeting
sequence (SEQ ID NO: 2)
gatctAGGGCTCTGCGTTTGCTCCAGGTAGTCCGCTGCTCCCTTG
GGCCTGGGCCCACTCACAGCCCTGGTGCCTCTGGCCGGCTGCACA
CCTCCTGGCGGGCAGCTOTGTTCTGCTCGAAATTGATGATGTGTT
CTGGCAATACCTGCATCATCATATCCGAGCAGAACACGGAGGCCT
OCCCTGACTGCCCACGOTGCCGTGGCCAAAGAGGATCTAAGGGCA
CCGCTGAGGGCCTACCTAACCATCOTGGGGAATAAGGACAGTGTC
ACCC
>SMN1 long promoter nucleic acid sequence
(SEQ ID NO: 3)
tcgaagctttataaaaacatacttttttttttacttttttttttt
tttctgagacacagcctcactctgtcgcccaggctggagtgcagg
tatcatgtttatctgtgagatgtacctttggcacattactttcct
gacatgagatttaaatttttttttttatcttgtgacaatttaact
tttttgacacataaaaattgtacatatttatttgtttgagatgga
gtcgcactctgtcactcaggctggagtgcagtggcgtgatcttgg
ctcactgcaacctccgcctcccgagttcaagtgattctcctggct
cagcctcccaagcagctgtcattacaggcctgcaccaccacaccc
ggctgattttgtatttttaggagaaacagggtttcaccatgttgg
gccaggctggtcttgaagtcctgacctcaagtgatccacccacct
tggcctcccaaagtgctgggattataggcatgagccaccgtacca
gacccctaaaaattgtatatatttaaggtgtaccatttgatgttt
agatatacattgtgaaatgattacattccacatattacctctaca
gagttaccatttttgtacacttggtcaacatcatcccattctccc
cttcctccacagatatttctgtatactatatagaagccaagggta
ttttgggggaagagctcaaagttcctttcgtggagttaaaaatat
atatatactatgtacatataagccatttagcaaccctagatgctt
aataaagaatactggaggcccggtgtggtggctcacacctgtaat
cccagcactttgggaggccgaggcggtcggattacgaggtcagga
gttcaagaccagcctggccaacatggtgaaaccccatctttacta
aaaatacaaaaattagccggggggggggggcctgtaatcccagct
actcggggggctgaggcagaattgcttgaacctgggaggcagagg
ttgcagtgagctgagatcacgccactgcattccagcctgggtgac
agagcaatattctgtcgcaaaaaaaaaaagaatactggaggctgg
gcgaggtggctcacacctgtaatcccagcattttgggatgccaga
ggcgggcggaatntcttgagctcaggagttcgagaccagcctaca
caatatgctccaaacgccgcttntacaaaacatacagaaactact
gggtgtggtggcgnncccctgtggtcctagatacttgggaggttg
aggcgggaggatcgcttgagctcgggaggtcgaggctgcaatgag
ccgagatggtgccactgcattctgacgacagagcgagattccgtt
tcaaaacaaacaacaaataaggttgggggatcaaatatcttctag
tgtttaaggatctgccttccttcctgcccccatgtttgtctttcc
ttgtttgtctttatatagatcaagcaggttttaaattcctagtag
gagcttacatttacttttccaagggggagggggaataaatatcta
cacacacacacacacacacacacacacacacacacacacacacac
acacacaccacactggagttcgagacgaggcctaagcaacatgcc
gaaaccccgtctctactaaatacaaaaaatagctgagcttggtgg
cgcacgcctatagtcctagctactggggaggctgaggtgggagga
tcgcttgagcccaagaagtcgaggctgcagtgagccgagatcgcg
ccgctgcactccagcctgagcgacagggcgaggctctgtctcaaa
acaaacaaacaaaaaaaaaaaggaaaggaaatataacacagtgaa
atgaaaggattgagagaaatgaaaaatatacacgccacaaatgtg
gggggcgataaccactcgtagatagcgtgagaagttactacaagc
ggtcctcccgggtaccgtactgttccgctcccagaagccccgggc
gccggaagtcgtcactcttaagaagggacggggccccacgctgcg
cacccgcgggtttgct
>SMN1 short promoter nucleic acid sequence
(SEQ ID NO: 4)
ggatgccagaggcgggcggaatntcttgagctcaggagttcgaga
ccagcctacacaatatgctccaaacgccgcttntacaaaacatac
agaaactacccgggtgtggtggcgnncccctgtggtcctagatac
ttgggaggttgaggcgggaggatcgcttgagctcgggaggtcgag
gctgcaatgagccgagatggtgccactgcattctgacgacagagc
gagattccgtttcaaaacaaacaacaaataaggttgggggatcaa
atatcttctagtgtttaaggatctgccttccttcctgcccccatg
tttgtctttccttgtttgtctttatatagatcaagcaggttttaa
attcctagtaggagcttacatttacttttccaagggggaggggga
ataaatatctacacacacacacacacacacacacacacacacaca
cacacacacacacacacaccacactggagttcgagacgaggccta
agcaacatgccgaaaccccgtctctactaaatacaaaaaatagct
gagcttggtggcgcacgcctatagtcctagctactggggaggctg
aggtgggaggatcgcttgagcccaagaagtcgaggctgcagtgag
ccgagatcgcgccgctgcactccagcctgagcgacagggcgaggc
tctgtctcaaaacaaacaaacaaaaaaaaaaaggaaaggaaatat
aacacagtgaaatgaaaggattgagagaaatgaaaaatatacacg
ccacaaatgtgggagggcgataaccactcgtagaaagcgtgagaa
gttactacaagcggtcctcccgggcaccgtactgttccgctccca
gaagccccgggcgccggaagtcgtcactcttaagaagggacgggg
ccccacgctgcgcacccggggtttgct
> SMN1 promoter (61 bp; SEQ ID NO: 5):
gccggaagtcgtcactcttaagaagggacggggccccacgctgcg
cacccgcgggtttgct
> SMN1 promoter (155 bp; SEQ ID NO: 6):
gtgggagggcgataaccactcgtagaaagcgtgagaagttactac
aagcggtcctcccgggcaccgtactgttccgctcccagaagcccc
gggcgccggaagtcgtcactcttaagaagggatggggccccacgc
tgcgcacccgcgggtttgct
> SMN1 promoter (303 bp; SEQ ID NO: 7):
gtgagccgagatcgcgccgctgcactccagcctgagcgacagcgc
gaggctctgtctcaaaacaaacaaacaaaaaaaaaaaggaaagga
aatataacacagtgaaatgaaaaggattgagagaaatgaaaaata
tacacgccacaaatgtggggggcgataaccactcgtagaaagcgt
gagaagttactacaagcggtcctcccgggcaccgtactgttccgc
tcccagaagccccgggcgccggaagtcgtcactcttaagaaggga
cggggccccacgctgcgcacccgcgggtttgct
>pAAVsc CB6 amiR-hSOD-1-X1
(SEQ ID NO: 8)
ctgcgcgctcgctcgctcactgaggccgcccgggcaaagcccggg
cgtcgggcgacctttggtcgcccggcctcagtgagcgagcgagcg
cgcagagagggagtgtagccatgctctaggaagatcaattcaatt
cacgcgtcgacattgattattgactAGCTctggtcgttacataac
ttacggtaaatggcccgcctggctgaccgcccaacgaccccCgcc
cattgacgtcaataatgacgtatgttcccatagtaacgccaatag
ggactttccattgacgtcaatgggggagtatttacggtaaactgc
ccacttggcagtacatcaagtgtatcatatgccaagtacgccccc
tattgacgtcaatgacggtaaatggcccgcctggcattatgccca
gtacatgaccttatgggactttcctacttggcagtacatctactc
gaggccacgttctgcttcactctccccatctcccccccctcccca
cccccaattttgtatttatttatttttaattattttgtgcagcga
tgggggcggggggggggggggggggggcgcgcgccaggcggggcg
gggcggggcgaggggcggggcggggcgaggcggagaggtgcggcg
gcagccaatcagcgcggcgcgctccgaaagtttccttttatggcg
aggcggcggcggcggcggccctataaaaagcgaagcgcgcggcgg
gcgggagcgggatcagccaccgcggtggcggcctagagtcgacga
ggaactgaaaaaccagaaagttaactggtaagtttagtctttttg
tcttttatttcaggtcccagatctAGGGCTCTGCGTTTGCTCCAG
GTAGTCCGCTGCTCCCTTGGGCCTGGGCCCACTGACAGCCCTGGT
GCCTCTGGCCGGCTGCACACCTCCTGGCGGGCAGCTGTGTTCTGC
TCGAAATTGATGATGTGTTCTGGCAATACCTGCATCATCATATCC
GAGCAGAACACGGAGGCCTGCCCTGACTGCCCACGGTGCCGTGGC
CAAAGAGGATCTAAGGGCACCGCTGAGGGCCTACCTAACCATCGT
GGGGAATAAGGACAGTGTCACCCggatccggtggtggtgcaaatc
aaagaactgctcctcagtggatgttgcctttacttctaggcctgt
acggaagtgttacttctgctctaaaagctgcggaattgtacccgc
ggccgatccaccggagcttatcgataccgtcgactagagctcgct
gatcagcctcgactgtgccttctagttgccagccatctgttgttt
gcccctcccccgtgccttccttgaccctggaaggtgccactccca
ctgtcctttcctaataaaatgaggaaattgcatcgcattgtctga
gtaggtgtcattctattctggggggggggtggggcaggacagcaa
gggggaggattgggaagacaatAGCCtaggtagataagtagcatg
gcgggttaatcattaactacaaggaacccctagtgatggagttgg
ccactccctctctgcgcgctcgctcgctcactgaggccgggcgac
caaaggtcgcccgacgcccgggctttgcccgggcggcctcagtga
gcgagcgagcgcgcag
>pAAVsc SMN1sp hsa-amiR-hSOD-1-X1
(SEQ ID NO: 9)
ctgcgcgctcgctcgctcactgaggccgcccgggcaaagcccggg
cgtcgggcgacctttggtcgcccggcctcagtgagcgagcgagcg
cgcagagagggagtgtagccatgctctaggaagatcaattcggta
caattcacgcgtggatgccagaggcgggcggaatntcttgagctc
aggagttcgagaccagcctacacaatatgctccaaacgccgcttn
tacaaaacatacagaaactacccggggggggcgnncccctgtggt
cctagatacttgggaggttgaggcgggggatcgcttgagctcggg
aggtcgaggctgcaatgagccgagatggtgccactgcattctgac
gacagagcgagattccgtttcaaaacaaacaacaaataaggttgg
gggatcaaatatcttctagtgtttaaggatctgccttccttcctg
cccccatgtttgtctttccttgtttgtctttatatagatcaagca
ggttttaaattcctagtaggagcttacatttacttttccaagggg
ggggggaataaatatctacacacacacacacacacacacacacac
acacacacacacacacacacacacaccacactggagttcgagaga
ggcctaagcaccatgccgaaaccccgtctctactaaatacaaaaa
atagctgagcttggtggcgcacgcctatagtcctagctactgggg
aggctgaggtgggaggatcgcttgagcccaagaagtcgaggctgc
agtgagccgagatcgcgccgctgcactccagcctgagcgacaggg
cgaggctctgtctcaaaacaaacaaacaaaaaaaaaaaggaaagg
aaatataacacagtgaaatgaaaggattgagagaaatgaaaaata
tacacgccacaaatgtgggagggcgataaccactcgtagaaagcg
tgagaagttactacaagcggtcctcccgggcaccgtactgttccg
ctcccagaagccccgggcgccggaagtcgtcactcttaagaaggg
acggggccccacgctgcgcacccgcgggtttgctccaccgcggtg
gTACcggccctagagtcgatcgaggaactgaaaaaccagaaagtt
aactggtaagtttagtctttttgtcttttatttcaggtcccagat
ctGGCAGCCTTGGAGTGGGTTCCTGCCCCCTCGGGCACACAAACA
GAGCTGAAGACCACCCTGGGCACCTCCTTGGCTGGCCGCATACCT
CCTGGCGGGCAGCTGTGtctgctcgaaattgatgatcTGTTCTGG
TGGTACCCACATCATCATATCCGAGCAGAACACAGAGGCCTGCCT
GGCCCTCGAGAGACTGCCCTGACTGAAGGCCCTATCAGGTGGGGG
AGGGGATCCTGATAGAGGGCACTGCTGCCACTGTTGGGGCCCAAG
Ctgcaggggatccggtggtggtgcaaatcaaagaactgctcctca
gtggatgttgcctttacttctaggcctgtacggaagtgttacttc
tgctctaaaagctgcggaattgtacccgcggccgatccaccggag
cttatcgataccgtcgactagagctcgctgatcagcctcgactgt
gccttctagttgccagccatctgttgtttgcccctcccccgtgcc
ttccttgaccctggaaggtgccactcccactgtcctttcctaata
aaatgaggaaattgcatcgcattgtctgagtaggtgtcattctat
tctggggggtggggggggcaggacagcaagggggaggattgggaa
gacaattaggtagataagtagcatggcgggttaatcattaactac
aaggaacccctagtgatggagttggccactccctctctgcgcgct
cgctcgctcactgaggccgggcgaccaaaggtcgcccgacgcccg
ggctttgcccgggcggcctcagtgagcgagcgagcgcgcag
>pAAVsc SMN1sp dual amiR-hSOD-1-X1
(SEQ ID NO: 10)
ctgcgcgctcgctcgctcactgaggccgcccgggcaaagcccggg
cgtcgggcgacctttggtcgcccggcctcagtgagcgagcgagcg
cgcagagagggagtgtagccatgctctaggaagatcaattcggta
caattcacgcgtggatgccagaggcgggcggaatntcttgagctc
aggagttcgagaccagcctacacaatatgctccaaacgccgcttn
tacaaaacatacagaaactacccgggtgtggtggcgnncccctgt
ggtcctagatacttgggaggttgaggcgggaggatcgcttgagct
cgggaggtcgaggctgcaatgagccgagatggtgccactgcattc
tgacgacagagcgagattccgtttcaaaacaaacaacaaataagg
ttgggggatcaaatatcttctagtgtttaaggatctgccttcctt
cctgcccccatgtttgtctttccttgtttgtctttatatagatca
agcaggttttaaattcctagtaggagcttacatttacttttccaa
gggggagggggaataaatatctacacacacacacacacacacaca
cacacacacacacacacacacacacacacaccacactggagttcg
agagaggcctaagcaacatgccgaaaccccgttctactaaataca
aaaaatagctgagcttggtggcgcacgcctatagtcctagctact
ggggaggctgaggtgggaggatcgcttgagcccaagaagtcgagg
ctgcagtgagccgagatcgcgccgctgcactccagcctgagcgac
agggcgaggctctgtctcaaaacaaacaaacaaaaaaaaaaagga
aaggaaatataacacagtgaaatgaaaggattgagagaaatgaaa
aatatacacgccacaaatgtgggagggcgataaccactcgtagaa
agcgtgagaagttactacaagcggtcctcccgggcaccgtactgt
tccgctcccagaagccccgggcgccggaagtcgtcactcttaaga
agggacggggccccacgctgcgcacccgcgggtttgctccaccgc
gctggTACcggccctagagtcgatcgaggaactgaaaaaccagaa
agttaactggtaagtttagtctttttgtcttttatttcaggtccc
agatctAGGGCTCTGCGTTTGCTCCAGGTAGTCCGCTGCTCCCTT
GGGCCTGGGCCCACTGACAGCCCTGGTGCCTCTGGCCGGCTGCAC
ACCTCCTGGCGGGCAGCTGTGTTCTGCTCGAAATTGATGATGTGT
TCTGGCAATACCTGCATCATCATATCCGAGCAGAACACGGAGGCC
TGCCCTGACTGCCCACUGTGCCTTGGCCAAAGAGGATCTAAGGGC
ACCGCTGAGGGCCTACCTAACCATCGTGGGGAATAAGGACAGTGT
CACCCtgcaggggatccggtggtggtgcaaatcaaagaactgctc
ctcagtggatgttgcctttacttctaggcctgtacggaagtgtta
cttctgctctaaaagctgcagaattgtacccgcggccgatccacc
ggGGCAGCCTTGGAGTGGGTTCCTGCCCCCTCGGGCACACAAACA
GAGCTGAAGACCACCCTGGGCACCTCCTTGGCTGGCCGCATACCT
CCTGGCGGGCAGCTGTGttctgctcgaaattgatgatgTGTTCTG
GTGGTACCCACATCATCATATCCGAGCAGAACACAGAGGCCTGCC
TGGCCCTCGAGAGACTGCCCTGACTGAAGGCCCTATCAGGTGGGG
GAGGGGATCCTGATAGAGGGCACTGCTGCCACTGTTGGGGCCCAA
Gaagcttatcgataccgtcgactagagctcgctgatcagcctcga
ctgtgccttctagttgccagccatctgttgtttgcccctcccccg
tgccttccttgaccctggaaggtggcactcccactgtcctttcct
aataaaatgaggaaattgcatcgcattgtctgagtaggtgtcatt
ctattctggggggtggggtggggcaggacagcaagggggaggatt
gggaagacaattaggtagataagtagcatggcgggttaatcatta
actacaaggaacccctagtgatggagttggccactccctctctgc
gcgctcgctcgctcactgaggccgggcgaccaaaggtcgcccgac
gcccgggctttgcccgggcggcctcagtgagcgagcgagcgcgca
g

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” second, “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A recombinant adeno-associated virus (rAAV) vector comprising a transgene comprising a human SMN1 promoter operably linked to a nucleic acid sequence encoding an artificial microRNA (amiRNA) targeting human SOD1, flanked by adeno-associated virus (AAV) inverted terminal repeats (ITRs).

2. The rAAV vector of claim 1, wherein the human SMN1 promoter comprises a nucleic acid sequence that is at least 70%, 80%, 90%, 95%, or 99% identical to the nucleic acid sequence set forth in any one of SEQ ID NOs: 3-7.

3. The rAAV vector of claim 1, wherein the endogenous SMN1 promoter comprises or consists of the nucleic acid sequence set forth in SEQ ID NO: 4.

4. The rAAV vector of claim 1, wherein the amiRNA comprises:
(i) a miR-33 prim-miRNA scaffold; and
(ii) a guide strand targeting a human SOD1 RNA transcript.

5. The rAAV vector of claim 1, wherein the amiRNA comprises or consists of the sequence set forth in SEQ ID NO: 1 or SEQ ID NO: 2.

6. The rAAV vector of claim 1, wherein the transgene is flanked by adeno-associated virus (AAV) inverted terminal repeats (ITRs).

7. The rAAV vector of claim 6, wherein the AAV ITRs are AAV2 ITRs.

8. The rAAV vector of claim 6, wherein at least one of the AAV ITRs is a mutant ITR (mTR).

9. A recombinant adeno-associated virus (rAAV) comprising:
(i) the rAAV vector of claim 1; and
(ii) at least one AAV capsid protein.

10. The rAAV of claim 9, wherein the at least one AAV capsid protein is selected from an AAV2, AAV3, AAV4, AAV5, AAV6, AAV7, AAV8, or AAV9 capsid protein, or a variant thereof.

11. The rAAV of claim 9, wherein the at least one AAV capsid protein is an AAV9 capsid protein.

12. A pharmaceutical composition comprising the rAAV vector or rAAV of claim 1, and a pharmaceutically acceptable excipient.

13. A method for delivering a transgene to a cell, the method comprising administering the rAAV vector of claim 1 to a cell

14. The method of claim 13, wherein the cell is a mammalian cell.

15. The method of claim 13, wherein the cell is a human cell.

16. The method of claim 13, wherein the cell is in a subject.

17. The method of claim 16, wherein the subject has or is suspected of having amyotrophic lateral sclerosis (ALS).

18. The method of claims 16, wherein the subject comprises a G93A mutation in a SOD1 gene.

19. A method for preventing or treating amyotrophic lateral sclerosis (ALS) in a subject, the method comprising administering the rAAV vector of claim 1 to the subject.

20. The method of claim 19, wherein the subject is a mammal.

21-30. (canceled)


SEQUENCE LISTING (10 sequences)

SEQ ID NO: 1 - DNA, 279 bp, synthetic construct
  source 1..279: mol_type = other DNA
gatctggcagccttggagtgggttcctgccccctcgggcacacaaacagagctgaagaccaccctgggcacctccttggctggccgcatacctcctggcgggcagctgtgttctgctcgaaattgatgatgtgttctggtggtacccacatcatcatatccgagcagaacacagaggcctgcctggccctcgagagactgccctgactgaaggccctatcaggtgggggaggggatcctgatagagggcactgctgccactgttggggcccaagctgca

SEQ ID NO: 2 - DNA, 274 bp, synthetic construct
  source 1..274: mol_type = other DNA
gatctagggctctgcgtttgctccaggtagtccgctgctcccttgggcctgggcccactgacagccctggtgcctctggccggctgcacacctcctggcgggcagctgtgttctgctcgaaattgatgatgtgttctggcaatacctgcatcatcatatccgagcagaacacggaggcctgccctgactgcccacggtgccgtggccaaagaggatctaagggcaccgctgagggcctacctaaccatcgtggggaataaggacagtgtcaccc

SEQ ID NO: 3 - DNA, 2050 bp, synthetic construct
  source 1..2050: mol_type = other DNA
tcgaagctttataaaaacatacttttttttttacttttttttttttttctgagacacagcctcactctgtcgcccaggctggagtgcaggttttcatgtttatctgtgagatgtacctttggcacattactttcctgacatgagatttaaatttttttttttatcttgtgacaatttaacttttttgacacataaaaattgtacatatttatttgtttgagatggagtcgcactctgtcactcaggctggagtgcagtggcgtgatcttggctcactgcaacctccgcctcccgagttcaagtgattctcctggctcagcctcccaagcagctgtcattacaggcctgcaccaccacacccggctgattttgtatttttaggagaaacagggtttcaccatgttgggccaggctggtcttgaagtcctgacctcaagtgatccacccaccttggcctcccaaagtgctgggattataggcatgagccaccgtaccagacccctaaaaattgtatatatttaaggtgtaccatttgatgtttagatatacattgtgaaatgattacattccacatattacctctacagagttaccatttttgtacacttggtcaacatcatcccattctccccttcctccacagatatttcttgtatactatatagaagccaagggtattttgggggaagagctcaaagttcctttcgtggagttaaaaatatatatatactatgtacatataagccatttagcaaccctagatgcttaataaagaatactggaggcccggtgtggtggctcacacctgtaatcccagcactttgggaggccgaggcggtcggattacgaggtcaggagttcaagaccagcctggccaacatggtgaaaccccatctttactaaaaatacaaaaattagccgggtgtggtggtgggcgcctgtaatcccagctactcggggggctgaggcagaattgcttgaacctgggaggcagaggttgcagtgagctgagatcacgccactgcattccagcctgggtgacagagcaatattctgtcgcaaaaaaaaaaagaatactggaggctgggcgaggtggctcacacctgtaatcccagcattttgggatgccagaggcgggcggaatntcttgagctcaggagttcgagaccagcctacacaatatgctccaaacgccgcttntacaaaacatacagaaactacccgggtgtggtggcgnncccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgct

SEQ ID NO: 4 - DNA, 928 bp, synthetic construct
  source 1..928: mol_type = other DNA
ggatgccagaggcgggcggaatntcttgagctcaggagttcgagaccagcctacacaatatgctccaaacgccgcttntacaaaacatacagaaactacccgggtgtggtggcgnncccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgct

SEQ ID NO: 5 - DNA, 61 bp, synthetic construct
  source 1..61: mol_type = other DNA
gccggaagtcgtcactcttaagaagggacggggccccacgctgcgcacccgcgggtttgct

SEQ ID NO: 6 - DNA, 155 bp, synthetic construct
  source 1..155: mol_type = other DNA
gtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgct

SEQ ID NO: 7 - DNA, 303 bp, synthetic construct
  source 1..303: mol_type = other DNA
gtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgct

SEQ ID NO: 8 - DNA, 1639 bp, synthetic construct
  source 1..1639: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccagatctagggctctgcgtttgctccaggtagtccgctgctcccttgggcctgggcccactgacagccctggtgcctctggccggctgcacacctcctggcgggcagctgtgttctgctcgaaattgatgatgtgttctggcaatacctgcatcatcatatccgagcagaacacggaggcctgccctgactgcccacggtgccgtggccaaagaggatctaagggcaccgctgagggcctacctaaccatcgtggggaataaggacagtgtcacccggatccggtggtggtgcaaatcaaagaactgctcctcagtggatgttgcctttacttctaggcctgtacggaagtgttacttctgctctaaaagctgcggaattgtacccgcggccgatccaccggagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - DNA, 1984 bp, synthetic construct
  source 1..1984: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtggatgccagaggcgggcggaatntcttgagctcaggagttcgagaccagcctacacaatatgctccaaacgccgcttntacaaaacatacagaaactacccgggtgtggtggcgnncccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgctccaccgcggtggtaccggccctagagtcgatcgaggaactgaaaaaccagaaagttaactggtaagtttagtctttttgtcttttatttcaggtcccagatctggcagccttggagtgggttcctgccccctcgggcacacaaacagagctgaagaccaccctgggcacctccttggctggccgcatacctcctggcgggcagctgtgttctgctcgaaattgatgatgtgttctggtggtacccacatcatcatatccgagcagaacacagaggcctgcctggccctcgagagactgccctgactgaaggccctatcaggtgggggaggggatcctgatagagggcactgctgccactgttggggcccaagctgcaggggatccggtggtggtgcaaatcaaagaactgctcctcagtggatgttgcctttacttctaggcctgtacggaagtgttacttctgctctaaaagctgcggaattgtacccgcggccgatccaccggagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 2254 bp, synthetic construct
  source 1..2254: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtggatgccagaggcgggcggaatntcttgagctcaggagttcgagaccagcctacacaatatgctccaaacgccgcttntacaaaacatacagaaactacccgggtgtggtggcgnncccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgctccaccgcggtggtaccggccctagagtcgatcgaggaactgaaaaaccagaaagttaactggtaagtttagtctttttgtcttttatttcaggtcccagatctagggctctgcgtttgctccaggtagtccgctgctcccttgggcctgggcccactgacagccctggtgcctctggccggctgcacacctcctggcgggcagctgtgttctgctcgaaattgatgatgtgttctggcaatacctgcatcatcatatccgagcagaacacggaggcctgccctgactgcccacggtgccgtggccaaagaggatctaagggcaccgctgagggcctacctaaccatcgtggggaataaggacagtgtcaccctgcaggggatccggtggtggtgcaaatcaaagaactgctcctcagtggatgttgcctttacttctaggcctgtacggaagtgttacttctgctctaaaagctgcggaattgtacccgcggccgatccaccggtggcagccttggagtgggttcctgccccctcgggcacacaaacagagctgaagaccaccctgggcacctccttggctggccgcatacctcctggcgggcagctgtgttctgctcgaaattgatgatgtgttctggtggtacccacatcatcatatccgagcagaacacagaggcctgcctggccctcgagagactgccctgactgaaggccctatcaggtgggggaggggatcctgatagagggcactgctgccactgttggggcccaagaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag